(19) United States
(12) Patent Application Publication
(10) Pub. No.: US 2019/0270801 A1
(43) Pub. Date: Sep. 5, 2019

(54) Oral Composition of Anti-TNF-Alpha Antibodies

(71) Applicant: Laboratoire Francais du Fractionnement et des Biotechnologies, Les Ulis (FR)

(72) Inventors: Christine GAUCHER, Sequedin (FR); Nadege HANDKE, Montrouge (FR); Cecile JAUME, Soisy sur Seine (FR)

(73) Assignee: Laboratoire Francais du Fractionnement et des Biotechnologies, Les Ulis (FR)

(21) Appl. No.: 16/320,297

(22) PCT Filed: Jul. 26, 2017
(86) PCT No.: PCT/EP2017/068918

§ 371 Date: Jan. 24, 2019

(30) Foreign Application Priority Data
Jul. 29, 2016 (FR) 1657442

(51) Int. Cl. C07K 16/24; A61K 9/16; A61K 9/19; A61P 37/06; A61P 29/00

(52) U.S. Cl. CPC C07K 16/241; A61K 9/1652; A61P 29/00; A61P 37/06; A61K 9/19

(57) ABSTRACT

The present application relates to a pharmaceutical composition for oral administration, comprising a monoclonal anti-tumor necrosis factor alpha antibody (TNFα) and at least one or more pharmaceutically acceptable excipients selected from the group comprising: a carboxymethyl-dextran, a chitosan, a cyclodextrin, or a combination of same, as well as the use of said composition in the treatment of inflammatory diseases or autoimmune diseases.

The present invention relates to compositions for the oral administration of an anti-tumour necrosis factor alpha (TNFα) antibody.

### TECHNOLOGICAL BACKGROUND

TNF alpha (or TNFα) is a pro-inflammatory cytokine that is secreted by, and interacts with, the cells of the immune system. TNFα has been shown to be involved in many human diseases, in particular chronic inflammatory diseases such as rheumatoid arthritis, Crohn's disease, ulcerative colitis, or also multiple sclerosis. A number of anti-TNFα antibodies are currently under development. Two antibodies are already on the market: infliximab (Remicade®), and adalimumab (Humira®), in subcutaneous or intravenous injectable forms.

However, a number of studies have reported that therapy using anti-TNFα antibodies, in particular systemically, may have unwanted secondary effects, in particular the occurrence of bacterial infections such as tuberculosis (Jarequi-Amezaga et al., Journal of Crohn's and Colitis, 2013, 7(3), pages 208-212), or listeria infections (Abreu et al., Journal of Crohn's and Colitis, 2013, 7(2), pages 175-182) or candida infections (Huang et al., Journal of Paediatric Gastroenterology and Nutrition, 2013, 56(4), pages 23-6).

This is why anti-TNFα antibody formulations for oral administration are currently under development. Nevertheless, none is on the market, and there is still a need to make available compositions allowing the efficacy and/or safety of anti-TNFα antibodies for oral administration to be improved.

A subject of the present invention is therefore a pharmaceutical composition for oral administration, comprising an anti-tumour necrosis factor alpha (TNFα) antibody and at least one or more pharmaceutically acceptable excipients selected from the group comprising: a carboxymethyl-dextran, a chitosan, a cyclodextrin or a combination thereof.

Within the meaning of the present invention, by “anti-TNF alpha antibody” or “anti-TNFα antibody” is meant any antibody binding specifically to human TNFα. Advantageously, the antibody is dissociated from human TNFα with the following constants: Kd less than 1×10⁻⁸ M (advantageously less than 1×10⁻⁹ M, more advantageously less than 1×10⁻¹⁰ M, even more advantageously less than 1×10⁻¹¹ M) and Koff of 1×10⁻³ s⁻¹ or less, both determined by a surface plasmon resonance test. Preferably, the antibody is a neutralizing antibody. In particular, it neutralizes the biological function of TNFα by blocking the interaction thereof with the TNF p55 and p75 receptors situated on the cell surface. The neutralization capacity of the antibody can be tested by a standard test, in particular by measuring the capacity of the antibody to neutralize the cytotoxicity of human TNFα on human fibroblast cells (L929), with a CI50 of 1×10⁻⁷ M or less, advantageously less than 1×10⁻⁸ M, more advantageously less than 1×10⁻⁹ M, or even more advantageously less than 1×10⁻¹⁰ M.

In an advantageous embodiment, the anti-TNF alpha antibody is a monoclonal antibody. The anti-TNF alpha antibody according to the invention can be an antibody from a mammal such as a mouse, or can be humanized, or also entirely human. In a particularly advantageous embodiment, the anti-TNF alpha antibody is a human monoclonal antibody.

In an advantageous embodiment, the anti-TNF alpha antibody according to the invention has a heavy chain comprising SEQ ID No.1 and a light chain comprising SEQ ID No.2.

SEQ ID NO: 1
MEFGLSWLFLVAILKGVQCEVQLVESGGGLVQPGRSLRLSCAASGFTFDD
YAMHWVRQAPGKGLEWVSAITWNSGHIDYADSVEGRFTISRDNAKNSLYL
QMNSLRAEDTAVYYCAKVSYLSTASSLDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK
SEQ ID NO: 2
MDMRVPAQLLGLLLLWLRGARCDIQMTQSPSSLSASVGDRVTITCRASQG
IRNYLAWYQQKPGKAPKLLIYAASTLQSGVPSRFSGSGSGTDFTLTISSL
QPEDVATYYCQRYNRAPYTFGQGTKVEIKRTVAAPSVFIFPPSDEQLKSG
TASVVCLLNNFYPREAKVQWKVDNALQSGNSQESVTEQDSKDSTYSLSST
LTLSKADYEKHKVYACEVTHQGLSSPVTKSFNRGEC

In an advantageous embodiment of the invention, the anti-TNF alpha antibody can be adalimumab, infliximab or golimumab. In an even more advantageous embodiment of the invention, the anti-TNF alpha antibody is adalimumab. Adalimumab is an immunoglobulin G (IgG) composed of two kappa light chains and two IgG1 heavy chains.

The antibody used in the present invention can be produced by any technique well known to a person skilled in the art. Advantageously, the antibody according to the invention is a recombinant antibody.

In a particular embodiment, the antibody can be produced by recombination in a host cell, transformed with one or more vector(s) that allow the expression and/or the secretion of the nucleotide sequences coding for the heavy chain and/or the light chain of the antibody. Generally, the vector comprises a promoter, translation initiation and termination signals, as well as suitable transcription regulatory regions. It is maintained in a stable fashion in the host cell and may optionally have particular signals that specify the secretion of the translated protein. These different elements are selected and optimized by a person skilled in the art as a function of the cell host used. Such vectors are prepared by methods commonly used by a person skilled in the art, and the resulting clones can be introduced into a suitable host by standard methods, such as lipofection, electroporation, the use of polycationic agents, heat shock, or chemical methods. The cell host can be selected from prokaryotic or eukaryotic systems, for example bacterial cells, but also yeast cells or animal cells, in particular cells from non-human mammals. The preferred mammal cells for the production of the monoclonal antibody are the YB2/0 rat cell line, the CHO hamster cell line, in particular the CHO dhfr- and CHO Lecl3, PER.C6TM (Crucell), 293, K562, NSO, SP2/0, BHK or COS cell lines. Insect cells may also be used. In a particularly advantageous embodiment, the anti-TNF alpha antibody is produced in mammary epithelial cells from a non-human mammal.

Another method of production is the expression of the recombinant antibody in transgenic organisms, for example in plants (Ayala M et al., Methods Mol. Biol., 2009; 483, pages 103-34.) or in the milk of transgenic animals such as the female rabbit, goat or pig (Pollock, D. P et al. Journal of Immunological Methods, 1999, 231, pages 147-157). In a particularly advantageous embodiment, the anti-TNF alpha antibody is produced in the milk of a transgenic non-human mammal.

According to a preferred embodiment, the antibody is produced in the milk of transgenic non-human mammals, genetically modified to produce this glycoprotein. The mammal can be for example a female goat, ewe, female bison, buffalo, camel, llama, mouse, rat, or also a cow, sow, female rabbit, or mare. Advantageously, the antibody is produced in the milk of a transgenic female goat.

Secretion of the antibody by the mammary glands, allowing the presence thereof in the milk of the transgenic mammal, involves controlling expression of the antibody in a tissue-dependent manner. Such control methods are well known to a person skilled in the art. Controlling expression is carried out by means of sequences allowing expression of the glycoprotein in a particular tissue of the animal. This involves in particular promoter sequences of the “WAP”, “β-casein”, and “β-lactoglobulin” type and optionally sequences of the signal peptide type. Advantageously, the antibody is produced in the mammary glands of a transgenic female goat, by using an expression vector comprising the sequence of the two chains, under the control of a 5′ β-casein promoter. A process of extraction of proteins of interest from the milk of transgenic animals is described in patent EP 0 264 166. Advantageously, more than 4 grams of antibody per litre of milk is produced, advantageously more than 5, 10, 15, 20, 25, 30, 35 grams per litre, even more advantageously, up to 70 grams per litre.

Advantageously, the antibody produced by animal transgenesis, in particular in the mammary glands of a transgenic female goat, is in the form of a population of anti-TNFα antibodies that have a glycosylation with a high level of galactosylation, for example greater than 60%, preferably greater than 70%, more preferably at least 80%. According to a particular aspect of the invention, the fucosylation level of the antibodies of the population overall is at least 50%, and in particular at least 60%.

According to a particular embodiment of the invention, the population of anti-TNFα antibodies comprises antibodies that comprise monogalactosylated N-glycans.

According to another particular embodiment of the invention, the population of anti-TNFα antibodies comprises antibodies that comprise bigalactosylated N-glycans.

According to another particular embodiment of the invention, the ratio of the galactosylation level of the antibodies of the population to the fucosylation level of the antibodies of the population is comprised between 1.0 and 1.4. According to another particular embodiment of the invention, at least 35% of the antibodies in the population comprises bigalactosylated N-glycans and at least 25% of the antibodies in the population comprises monogalactosylated N-glycans.

According to another particular embodiment of the invention, the sialylation level of the antibodies is at least 50%, preferably at least 70%, or even at least 90%.

According to another particular embodiment of the invention, the antibodies are completely sialylated.

The biosynthesis of the N-glycans is not regulated by a codification, as is the case with the proteins, but is mainly dependent on the expression and the activity of the specific gylcosyltransferases in a cell. Thus, a glycoprotein, such as the Fc fragment of an antibody, normally exists as a heterogenous population of glycoforms that bear different glycans on the same protein skeleton.

A highly galactosylated antibody population is an antibody population in which the galactosylation level of the antibodies of the population overall is at least 50%, at least 60%, at least 70%, at least 80%, at least 90%, up to 100% galactosylation.

According to a particular method for the population of highly galactosylated antibodies, the galactosylation level of the antibodies of the population overall is at least 60%.

The galactosylation level can be determined with the following formula:

$${\sum }_{i = 1}^{n} \frac{( Galactose index )}{( A index )} \times ( \% relative surface area )$$

in which:

“n” represents the number of N-glycan peaks analyzed on a chromatogram, for example from a normal-phase high-performance liquid chromatography (NP-HPLC) spectrum.

“Galactose Index” represents the number of galactose units on the antenna of the glycan corresponding to the peak,

“A Index” represents the number of N-acetyl-glucosamine units on the antenna of the glycan form corresponding to the peak (excluding the two N-acetyl-glucosamine units of the common skeleton structure of the glycans), and

“% relative surface area” corresponds to the percentage of the area under the corresponding peak.

The galactosylation level of the antibodies of the antibody population can be determined, for example, by releasing the N-glycans from the antibodies, resolving the N-glycans on a chromatogram, identifying the oligosaccharide unit of the N-glycan that corresponds to a specific peak, determining the intensity of the peak, and applying the data to the aforementioned formula.

Antibodies that are galactosylated include antibodies that have monogalactosylated and bigalactosylated N-glycans.

In a particular embodiment, the population of highly galactosylated antibodies comprises antibodies that comprise monogalactosylated N-glycans, which may or may not be sialylated. According to a particular aspect of the highly galactosylated antibody population, at least 1%, at least 5%, at least 10%, at least 15%, at least 20%, at least 25%, at least 30%, at least 40%, at least 50%, at least 60%, at least 70%, at least 80%, at least 90%, up to 100% of the N-glycans of the antibodies comprise monogalactosylated N-glycans.

According to another particular embodiment of the invention, in the highly galactosylated antibody population, at least 25% of the antibodies comprise monogalactosylated N-glycans.

According to a particular aspect of the highly galactosylated antibody population, the population comprises antibodies that comprise bigalactosylated N-glycans, which may or may not be sialylated. According to a particular aspect of the highly galactosylated antibody population, at least 1%, at least 5%, at least 10%, at least 15%, at least 20%, at least 25%, at least 30%, at least 40%, at least 50%, at least 60%, at least 70%, at least 80%, at least 90%, up to 100% of the N-glycans of the antibodies comprise bigalactosylated N-glycans.

According to another particular embodiment of the invention, in the highly galactosylated antibody population, at least 35% of the antibodies comprise bigalactosylated N-glycans.

According to yet another aspect of the highly galactosylated antibody population, the population comprises antibodies that comprise monogalactosylated N-glycans, which may or may not be sialylated, and antibodies that comprise bigalactosylated N-glycans, which may or may not be sialylated.

According to a particular aspect of the highly galactosylated antibody population, at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise monogalactosylated N-glycans, and at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise bigalactosylated N-glycans.

According to another particular aspect of the highly galactosylated antibody population, at least 25% of the antibodies in the population comprises monogalactosylated N-glycans, and at least 35% of the antibodies comprise bigalactosylated N-glycans.

In a particularly advantageous embodiment, the anti-TNFα antibody is produced by the mammary epithelial cells of a transgenic non-human mammal and is produced exogenously in the mammary gland. The therapeutic antibodies thus produced have a high galactosylation level, and optionally increased levels of terminal bonds of alpha-2,6 sialic acid on the glycan residues thereof bound to the Fc fragment.

In certain embodiments, the antibody has a high-mannose glycosylation profile. By “high-mannose glycosylation profile” as used here is meant an antibody that contains at least one oligomannose or an antibody composition in which at least 30% of the antibody contains at least one oligomannose. In certain embodiments at least 30%, 40%, 50%, 60%, 70%, 80%, 90% or more of the sugars of the antibodies are non-fucosylated oligomannoses. In other embodiments less than 50%, 40%, 30%, 20%, 10%, 5%, or less of the sugars of the antibodies contain fucose. In another embodiment, the antibodies have a low fucose content and a high oligomannose content. Thus, in other embodiments, at least 30%, 40%, 50%, 60%, 70%, 80% or 90% or more of the sugars of the antibodies are of oligomannose and less than 50%, 40%, 30%, 20%, 10% or 5% of the sugars of the antibodies contain fucose. Thus, in another embodiment, at least 30%, 40%, 50%, 60%, 70%, 80% or 90% or more of the sugars of the antibodies are non-fucosylated oligomannose and less than 50%, 40%, 30%, 20%, 10% or 5% of the sugars of the antibodies contain fucose.

According to a particular aspect, the oligosaccarides containing mannose can advantageously contain from 5 to 9 mannoses. An oligosaccharide containing Y mannoses is denoted by the term ManY. For example, the oligosaccharides containing mannose can include Man5, Man6, Man7, Man8 et Man9. In certain embodiments, the antibody, such as Adalimumab, produced transgenically has a high Man6 content. In certain embodiments, the major sugar is Man5. In certain embodiments at least 10%, 15% or more of the sugars are Man5. Advantageously, at least 20% of the sugars are Man5. In other embodiments, the major sugar is Man6. In certain embodiments, at least 10%, 15% or more of the sugars of the antibody produced transgenically are Man6. Advantageously, at least 20% of the sugars are Man6. In other embodiments, the major sugar is Man7. In certain embodiments, at least 10%, 15% or more of the sugars are Man7. Advantageously, at least 20% of the sugars are Man7.

The antibodies that have a high-galactose or -mannose profile, as described above, are particularly advantageous, in that they have a high affinity for the FcγRIIIa (CD16) receptor. By “high affinity” is meant an affinity at least equal to 2×10⁶ M⁻¹, preferably at least equal to 2×10⁷ M⁻¹, 2×10⁸ M⁻¹ or 2×10⁹ M⁻¹, as determined by Scatchard analysis or BIAcore technology (Label-free surface plasmon resonance-based technology). This receptor is found on a large number of immune cells, among which are the natural killer cells, the macrophages, neutrophils and mast cells.

This affinity for CD 16 allows an improvement in the complement-dependent cytotoxicity (CDC) or antibody-dependent cell-mediated cytotoxicity (ADCC) activities, or also phenomena of phagocytosis of the target cells, with respect to antibodies that are not highly galactosylated or not highly mannosylated. In certain embodiments, the populations of anti-TNFα antibodies produced in epithelial cells from mammary glands are superior, in terms of binding to the soluble TNFα, to the antibodies produced in cells that are not epithelial cells from mammary glands. In certain embodiments, the populations of anti-TNFα antibodies produced in cells from mammary glands are superior, in terms of binding to the transmembrane TNFα, to the antibodies produced in cells that are not epithelial cells from mammary glands. Tests for determining the level of binding to the soluble TNFα or the transmembrane TNFα are well established (see for example Horiuchi et al., Rheumatology et al. 49, page 1215).

In a particularly advantageous embodiment, the anti-TNFα antibody can be the antibody described in international application WO2014/125374.

In a particular embodiment, the pharmaceutical composition of the invention as defined above is devoid of at least one of the following excipients: caprylic acid or a caprylate salt, glucose, fructose, galactose, mannose, sorbose, saccharose, ribose, deoxyribose, sucrose, maltitol, inulin, lecithin, trehalose, lactose, maltose, raffinose, mannitol, sorbitol, glycerol, arabitol, lactitol, xylitol, polypropylene glycol, polyethylene glycol, polyoxyethylene sorbitan fatty acid ester, polysorbate 20, polysorbate 80, poloxamers, polyoxyethylene alkyl ethers, alkyl phenyl polyoxyethylene ethers, polyoxyethylene-polyoxypropylene copolymer, sodium dodecyl sulphate, sodium succinate, sodium chloride, sodium citrate, citric acid monohydrate, sodium phosphate dibasic dihydrate, sodium phosphate monobasic dihydrate, sodium phosphate monobasic monohydrate or EDTA.

Within the meaning of the present invention, by the term “devoid of” is meant that the composition of the invention does not contain any caprylic acid or caprylate salt, glucose, fructose, galactose, mannose, sorbose, saccharose, ribose, deoxyribose, sucrose, maltitol, inulin, lecithin, trehalose, lactose, maltose, raffinose, mannitol, sorbitol, glycerol, arabitol, lactitol, xylitol, polypropylene glycol, polyethylene glycol, polyoxyethylene sorbitan fatty acid ester, polysorbate 20, polysorbate 80, poloxamers, polyoxyethylene alkyl ethers, alkyl phenyl polyoxyethylene ethers, polyoxyethylene-polyoxypropylene copolymer, sodium dodecyl sulphate, sodium succinate, sodium chloride, sodium citrate, citric acid monohydrate, sodium phosphate dibasic dihydrate, sodium phosphate monobasic dihydrate, sodium phosphate monobasic monohydrate or EDTA, or that they are present in a negligible quantity, in particular a quantity less than 0.001% (m/m).

In a particularly advantageous embodiment, the pharmaceutical composition of the invention as defined above is devoid of any molecule with an anticoagulant effect, in particular EDTA.

In another particularly advantageous embodiment, the pharmaceutical composition of the invention as defined above is devoid of any surface active agent, in particular polysorbate 20, polysorbate 80 or poloxamer.

In another particularly advantageous embodiment, the pharmaceutical composition of the invention as defined above is devoid of any molecule with a laxative effect, in particular mannitol, sorbitol, glycerol, lactitol or xylitol.

The aforementioned excipients were ruled out in particular due to their anticoagulant activity, such as in particular EDTA, or due to the fact that they would present a risk of developing Crohn's disease when ingested, such as in particular polysorbate 20, polysorbate 80 or poloxamer, or due to the fact that these excipients can have a laxative effect, such as in particular mannitol, maltitol, lactitol, xylitol or sorbitol.

In a particularly advantageous embodiment of the invention, the pharmaceutical composition for oral administration comprises:
a monoclonal anti-tumour necrosis factor alpha (TNF-alpha) antibody as active ingredient, and
carboxymethyl-dextran (CMD) as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
chitosan, as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
a cyclodextrin, as pharmaceutically acceptable excipient.

In an advantageous embodiment of the invention, the cyclodextrin can be selected from α-cyclodextrin, β-cyclodextrin, γ-cyclodextrin, a derivative thereof, such as in particular a hydroxypropyl, hydroxyethyl, ethyl or methyl derivative, a sulphobutyl ether beta-cyclodextrin, a branched cyclodextrin, a cyclodextrin-based polymer or a mixture thereof. Advantageously, the cyclodextrin can be hydroxypropyl-β-cyclodextrin (or HPBCD).

In a particularly advantageous embodiment of the invention, the pharmaceutical composition for oral administration comprises:
a monoclonal anti-tumour necrosis factor alpha (TNF-alpha) antibody as active ingredient, and
hydroxypropyl-β-cyclodextrin (HPBCD), as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
a combination of carboxymethyl-dextran (CMD), and cyclodextrin, in particular hydroxypropyl-β-cyclodextrin (HPBCD), as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
a combination of carboxymethyl-dextran (CMD), and chitosan, as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
a combination of a cyclodextrin, in particular hydroxypropyl-β-cyclodextrin (HPBCD) and chitosan, as pharmaceutically acceptable excipient.

In another particularly advantageous embodiment of the invention, the pharmaceutical composition for oral administration comprises:
a monoclonal anti-tumour necrosis factor alpha (TNF-alpha) antibody as active ingredient, and
a combination of carboxymethyl-dextran (CMD), cyclodextrin, in particular hydroxypropyl-β-cyclodextrin (HPBCD) and chitosan, as pharmaceutically acceptable excipient.

In a particularly advantageous embodiment of the invention, the pharmaceutical composition for oral administration can also comprise at least one amino acid. Within the meaning of the present invention, the amino acid can be selected from arginine, glycine, lysine, histidine, glutamate, glutamine, asparagine, isoleucine, leucine, alanine, phenylalanine, threonine, tyrosine, tryptophan, methionine, serine, proline, cysteine, selenocysteine, proline, valine, a salt derivative thereof, such as in particular a hydrochloride or a phosphate, or a mixture thereof. Advantageously, the amino acid is selected from the hydrophilic amino acids. In an advantageous embodiment, the amino acid is glycine or one of the salt derivatives thereof, such as glycine hydrochloride. In another particularly advantageous embodiment, the amino acid is arginine or one of the salt derivatives thereof, such as arginine hydrochloride.

In a particularly advantageous embodiment of the invention, the pharmaceutical composition for oral administration comprises:
a monoclonal anti-tumour necrosis factor alpha (TNF-alpha) antibody as active ingredient,
carboxymethyl-dextran (CMD) as pharmaceutically acceptable excipient, and
an amino acid, in particular glycine, optionally in the hydrochloride form thereof.

In a particularly advantageous embodiment of the invention, the pharmaceutical composition for oral administration comprises:
a monoclonal anti-tumour necrosis factor alpha (TNF-alpha) antibody as active ingredient,
chitosan, as pharmaceutically acceptable excipient, and
an amino acid, in particular glycine, optionally in the hydrochloride form thereof.

In a particularly advantageous embodiment of the invention, the pharmaceutical composition for oral administration comprises:
a monoclonal anti-tumour necrosis factor alpha (TNF-alpha) antibody as active ingredient,
a cyclodextrin, in particular hydroxypropyl-β-cyclodextrin (HPBCD), as pharmaceutically acceptable excipient,
an amino acid, in particular glycine, optionally in the hydrochloride form thereof.

In a particularly advantageous embodiment of the invention, the pharmaceutical composition for oral administration comprises:
a monoclonal anti-tumour necrosis factor alpha (TNF-alpha) antibody as active ingredient,
a combination of carboxymethyl-dextran (CMD) and a cyclodextrin, in particular hydroxypropyl-β-cyclodextrin (HPBCD), as pharmaceutically acceptable excipient, and
an amino acid, in particular glycine, optionally in the hydrochloride form thereof.

In a particularly advantageous embodiment of the invention, the pharmaceutical composition for oral administration comprises:
a monoclonal anti-tumour necrosis factor alpha (TNF-alpha) antibody as active ingredient,
a combination of carboxymethyl-dextran (CMD) and chitosan, as pharmaceutically acceptable excipient, and
an amino acid, in particular glycine, optionally in the hydrochloride form thereof.

In a particularly advantageous embodiment of the invention, the pharmaceutical composition for oral administration comprises:
a monoclonal anti-tumour necrosis factor alpha (TNF-alpha) antibody as active ingredient,
a combination of a cyclodextrin, in particular hydroxypropyl-β-cyclodextrin (HPBCD) and chitosan, as pharmaceutically acceptable excipient, and
an amino acid, in particular glycine, optionally in the hydrochloride form thereof.

In another particularly advantageous embodiment of the invention, the pharmaceutical composition for oral administration consists of:
a monoclonal anti-tumour necrosis factor alpha (TNF-alpha) antibody as active ingredient, and
carboxymethyl-dextran (CMD) as pharmaceutically acceptable excipient.

In another particularly advantageous embodiment of the invention, the pharmaceutical composition for oral administration consists of:
a monoclonal anti-tumour necrosis factor alpha (TNF-alpha) antibody as active ingredient,
carboxymethyl-dextran (CMD), and
a cyclodextrin, in particular hydroxypropyl-β-cyclodextrin (HPBCD).

In another particularly advantageous embodiment of the invention, the pharmaceutical composition for oral administration consists of:
a monoclonal anti-tumour necrosis factor alpha (TNF-alpha) antibody as active ingredient,
a combination of carboxymethyl-dextran (CMD) and a cyclodextrin, in particular hydroxypropyl-β-cyclodextrin (HPBCD), as pharmaceutically acceptable excipient,
an amino acid, in particular glycine, optionally in the hydrochloride form thereof.

In another particularly advantageous embodiment of the invention, the pharmaceutical composition for oral administration consists of:
a monoclonal anti-tumour necrosis factor alpha (TNF-alpha) antibody as active ingredient, and
chitosan, as pharmaceutically acceptable excipient.

In a particularly advantageous embodiment of the invention, the pharmaceutical composition according to the invention is in solid form. The pharmaceutical composition in solid form can be obtained by any technique well known to a person skilled in the art. Advantageously, the pharmaceutical composition in solid form can be obtained by freeze drying, spray drying or by evaporation drying.

The compositions of the present invention can be presented in all pharmaceutical forms normally used for oral administration, in particular in the form of tablets, gelatin capsules, capsules, lozenges, powder, syrup or any form for a solid oral preparation or any form of drinkable preparation.

In a particularly advantageous embodiment of the invention, the composition is presented in the form of a sustained-release and/or delayed-release formulation. Within the meaning of the present invention, by “sustained-release and/or delayed-release formulation” is meant a pharmaceutical form suitable for a targeted release at the level of the gastro-intestinal tract, and in particular the intestine. By “intestine” is meant here all the parts of the intestine, in particular the colon. Such compositions are particularly useful in the treatment of inflammatory diseases of the intestine, as they allow local activity at the site of infection (in particular the small intestine or colon). They also limit the passage of the antibodies into the bloodstream, limiting the side effects associated with the anti-TNFα antibodies.

Several strategies exist for preparing medicaments for oral administration, the active ingredient(s) of which are only released at the level of the intestine, preferably at the level of the colon. Certain strategies comprise covalent bonding of the medicament with a support. Excipients and vehicles that are degraded by the bacteria of the colon can also be used. All these strategies are well known to a person skilled in the art.

In a particular embodiment, the pharmaceutical composition in the form of a sustained-release and/or delayed-release formulation comprising the anti-TNFα antibody and at least one pharmaceutically acceptable excipient such as described above can be formulated in solid dosage forms such as tablets or gelatin capsules. Advantageously, the pharmaceutical composition in the form of a sustained-release and/or delayed-release formulation comprising the anti-TNFα antibody and at least one pharmaceutically acceptable excipient such as described above can also comprise at least one coating agent. By means of the resistance thereof to the acid pH of the stomach (comprised between 1 and 3) the coating agent makes it possible in particular to protect the composition comprising the anti-TNFα antibody and at least one pharmaceutically acceptable excipient against the acid pH of the stomach and release thereof in the small intestine and the colon, where the pH values are close to 7. Advantageously, the coating agent can be a film-forming agent, preferably gastric-soluble, or a sugar-coating agent. In a particularly advantageous embodiment, the coating agent can be selected from the group comprising: acrylic acid derivatives, methacrylic acid derivatives, ethyl acrylate derivatives, hydroxypropyl methyl cellulose derivatives, polyvinyl acetate phthalate derivatives; poly(methyl acrylate-co-methyl methacrylate-co-methacrylic acid), poly(methacrylate acid-co-methyl methacrylate), in particular marketed under the names Eudragit® S100, Eudragit® FS30D, Eudragit® L100, Eudragit®L12.5, Eudragit® L30D-55, Eudragit®, L100-55, Eudragit® S12.5, hydroxypropyl methylcellulose phthalate (HPMCP), hydroxypropyl methylcellulose acetate succinate, also called HPMCAS and marketed under the name AQOAT®, Shellac or a mixture thereof.

In a particular embodiment, the pharmaceutical composition in the form of a sustained-release and/or delayed-release formulation comprising the anti-TNFα antibody and at least one pharmaceutically acceptable excipient such as described above can comprise a monolayer of a single coating agent, a monolayer constituted by a mixture of several coating agents, a multilayer constituted by a single coating agent, a multilayer constituted by a mixture of several different coating agents or a superimposition of several monolayers constituted by a single coating agent.

In another embodiment, formulations can be used that are coated with polymers degradable by the microorganisms of the colon (more particularly by the bacterial enzymes such as azoreductases and glycosidases), for example azoic polymers having a high degree of hydrophilia. Gels and hydrogels can also be used, in particular polysaccharide-based hydrogels.

The compositions of the invention can comprise or be combined with other therapeutic agents useful in the treatment of inflammatory diseases or auto-immune diseases.

Another aspect of the invention relates to a process for the preparation of a sustained-release and/or delayed-release pharmaceutical composition, said process comprising:

a) A step of mixing anti-TNF alpha antibody with one or more pharmaceutically acceptable excipients selected from the group comprising: a carboxymethyl-dextran, a chitosan, a cyclodextrin or a combination thereof;

b) A step of drying the composition obtained in step a), particularly by spraying;

c) A step of coating.

Advantageously, coating step c) can be carried out by any technique well known to a person skilled in the art. In a particular embodiment, coating step c) can be carried out by sugar-coating, film-coating, in particular by the addition of film-forming agents, by spraying, enteric coating in a fluidized bed, dry coating, layering. By “dry coating” is meant any process without solvent using a mechanical action to cover a solid pharmaceutical form with a discrete or continuous layer of inert or active powders. It makes it possible, for example, to obtain composite particles comprising several layers of different compositions.

In a particular embodiment, coating step c) can be carried out with one or more coating agents by applying:
a monolayer constituted by a single coating agent, or
a monolayer constituted by a mixture of several coating agents, or
a multilayer constituted by a single coating agent, i.e. a superimposition of several monolayers constituted by the same coating agent, or
a multilayer constituted by a mixture of several coating agents, or
a superimposition of several monolayers constituted by a single coating agent, each of the monolayers having a different coating agent.

Another aspect of the invention relates to a sustained-release and/or delayed-release pharmaceutical composition comprising an anti-tumour necrosis factor alpha (TNFα) antibody and at least one or more pharmaceutically acceptable excipients selected from the group comprising: a carboxymethyl-dextran, a chitosan, a cyclodextrin or a combination thereof, for use thereof in the treatment of inflammatory diseases or auto-immune diseases.

Another aspect of the invention is a method for the treatment of inflammatory diseases or auto-immune diseases comprising the administration to a patient having need thereof of a sustained-release and/or delayed-release pharmaceutical composition comprising an anti-tumour necrosis factor alpha (TNFα) antibody and at least one or more pharmaceutically acceptable excipients selected from the group comprising: a carboxymethyl-dextran, a chitosan, a cyclodextrin or a combination thereof.

### FIGURES

FIG. 1: Results of the functional test at T0, T+2 hours and T+5 hours after passing through the SHIME® system.

In this figure, the 10 formulations are presented in the following order (from left to right): 1/without excipients, 2/HPBCD, 3/CMD, 4/HPBCD+CMD, 5/HPCD+EDTA, 6/Polysorbate 20+EDTA+Trehalose, 7/Poloxamer 188+EDTA+Trehalose, 8/CMD+Polysorbate 20+Arginine, 9/CMD+Polysorbate 20+Glycine, 10/Chitosan.

FIG. 2: Characteristics of the different formulations tested in Example 4.

The following examples illustrate the invention without limiting the scope thereof.

### EXAMPLES

In the examples below, unless otherwise stated, the anti-TNFα antibodies used are those described in the Applicant's international application WO2014/125374.

### Example 1: Pre-Selection of Excipients by Compatibility Test

A 1^{st} test was conducted in order to preselect several excipients on the basis of the satisfactory compatibility thereof with the anti-TNFα antibodies at 1 mg/mL in 10 mM acetate buffer at pH 5.5.

The excipients tested are listed in the following table:

TABLE 1
formulations tested in compatibility test
Excipients

| | | |
|---|---|---|
| Human serum albumin | 1.25 | mg/mL |
| Arginine | 1.25 | mg/mL |
| Carbopol 971P | 1.25 | mg/mL |
| Carbopol 974P | 1.25 | mg/mL |
| Carboxymethyl-Dextran | 20 | mg/mL |
| Chitosan | 1.25 | mg/mL |
| Glycine | 1.25 | mg/mL |
| Hydroxypropyl-beta-cyclodextrin (HPBCD) | 20 | mg/mL |
| Inulin | 20 | mg/mL |
| Lecithin | 1.25 | mg/mL |
| Maltitol | 20 | mg/mL |
| Mannitol | 20 | mg/mL |
| Na2 EDTA | 1.25 | mg/mL |
| Pluronic F127 | 1.25 | mg/mL |
| Poloxamer 188 | 1.25 | mg/mL |
| Polysorbate 20 | 1.25 | mg/mL |
| Sodium lauryl sulphate (SLS) | 1.25 | mg/mL |
| Sorbitol | 20 | mg/mL |

In the presence of the different excipients, the formulations of anti-TNFα antibodies are stability-tested at 5, 40 and 50° C. for 3, 7 and 14 days.

At each stage, the formulations are subjected to the following analyses:
SEC (Size Exclusion Chromatography) allowing analysis of the monomer forms, high molecular weight forms and degraded forms of the anti-TNα antibody. This analysis shows the capacity of the formulation to preserve the protein in the form of interest (active monomer form) thereof as well as the formation of polymerized forms and/or aggregates (high molecular weight forms that may be responsible for side effects when administered to the patient) and/or the formation of inactive degraded forms.
   Simplified protocol: the samples of anti-TNFα antibody formulation are diluted to 2 mg/mL in a PBS buffer (10 mM Na₂HPO₄+1.8 mM KH₂PO₄+2.7 mM KCl+137 mM NaCl) then centrifuged for 5 min at 15,000 g. The supernatants are transferred to the flasks, placed in chromatography gel for separation, then each peak is detected by measuring the absorbance at 280 nm.
DLS (Dynamic Light Scattering) allowing the aggregation of a formulation to be monitored. This analysis shows the capacity of the formulation to preserve the protein in the form of interest (active monomer form) thereof as well as the formation of aggregates (high molecular weight forms that may be responsible for side effects when administered to the patient, or loss of efficacy).
   Simplified protocol: The samples are diluted to 2 mg/mL in ultrapure water, then analyzed by DLS.
Functional test: The functional activity test consists of a test of the binding of the antibody to TNFα membrane proteins, with detection by flow cytometry, making it possible to determine the preservation of the binding activity of the antibody to the target thereof.
   Simplified protocol: 2×10⁵ Jurkat cells transfected to express the membrane TFNα are incubated with 100 μl of anti-TNF antibodies at different concentrations (0 to 100 μl/ml, final concentration) at 4° C. for 30 minutes. After washing, the goat anti-IgG Fc antibody coupled to phycoerythrin (100 μl of a 1:100 dilution) is added at 4° C. over 30 minutes. The cells are washed and the mean fluorescence intensity (MFI) is measured by flow cytometry. A response curve is established with the reference antibody (anti-TNFα antibody in PBS buffer) in order to define the minimum antibody concentration giving the plateau. All the samples are tested at 0.125 μg/ml. The results are expressed as a percentage with respect to a reference and normalized as follows:
Preserved activity=80-100% of initial activity
Activity slightly reduced: 50-80% of initial activity
Loss of activity: <50% of initial activity

### The Results are as Follows:

### Results of SEC

The results of SEC analysis are presented in Table 2:

TABLE 2
Results of SEC analysis

| | | | Results of SEC | Results of SEC |
|---|---|---|---|---|
| | | | analysis - 14 | analysis - 14 |
| Excipient | | | days at 5° C. | days at 50° C. |
| Human | 1.25 | mg/mL | Average | Average |
| serum | | | | |
| albumin | | | | |
| Arginine | 1.25 | mg/mL | Good | Good |
| Carbopol | 1.25 | mg/mL | NA - precipitation | NA - precipitation |
| 971P | | | of the protein | of the protein |
| Carbopol | 1.25 | mg/mL | NA - precipitation | NA - precipitation |
| 974P | | | of the protein | of the protein |
| Carboxy- | 20 | mg/mL | Good | Good |
| methyl- | | | | |
| Dextran | | | | |
| (CMD) | | | | |
| Chitosan | 1.25 | mg/mL | NA - unsuitable | NA - unsuitable |
| | | | analysis | analysis |
| | | | conditions | conditions |
| | | | (optimum pH) | (optimum pH) |
| Glycine | 1.25 | mg/mL | Good | Good |
| Hydroxy- | 20 | mg/mL | Good | Good |
| propyl-beta- | | | | |
| cyclodextrin | | | | |
| Inulin | 20 | mg/mL | Precipitation of | Precipitation of |
| | | | the protein | the protein |
| Lecithin | 1.25 | mg/mL | Precipitation of | Precipitation of |
| | | | the protein | the protein |
| Maltitol | 20 | mg/mL | Slight precipitation | Slight precipitation |
| | | | of the protein | of the protein |
| Mannitol | 20 | mg/mL | Good | Good |
| Na2 EDTA | 1.25 | mg/mL | Good | Good |
| Pluronic | 1.25 | mg/mL | Good | Good |
| F127 | | | | |
| Poloxamer | 1.25 | mg/mL | Good | Good |
| 188 | | | | |
| Poly- | 1.25 | mg/mL | Good | Good |
| sorbate 20 | | | | |
| Sodium | 1.25 | mg/mL | Precipitation of | Precipitation of |
| lauryl | | | the protein | the protein |
| sulphate | | | | |
| (SLS) | | | | |
| Sorbitol | 20 | mg/mL | Good | Good |

Results considered “Good” allow both the quantity and the quality of the protein to be preserved, in particular after stress at 50° C. for 14 days (preservation of the quantity of proteins and preservation of the distribution profile of the forms of the protein).

The total level of proteins is significantly reduced for the formulations comprising Carbopol, lecithin, SLS, albumin, inulin and maltitol, indicating the formation of insoluble proteins that were removed by centrifugation.

Generally, no variation in the quantity of protein and in the distribution of the different forms of the protein is observed for the other formulations tested, showing that these formulations are compatible with the anti-TNFα antibody.

### Results of DLS

The results of DLS are presented in Table 3:

TABLE 3
Results of SEC analysis - results of intensity distribution at 5° C. on day 5

| | Results of intensity distribution | | | | | |
|---|---|---|---|---|---|---|
| | Average size | | | | | |
| | (diameter in nm) | | | Intensity (%) | | |
| | | | | Peak 1 | | |
| | Peak 1: | Peak 2 | Peak 3 | Mono- | Peak 2 | Peak 3 |
| Excipient | monomers | (others) | (others) | mers | (others) | (others) |
| Human | 12.5 | 292.7 | 0.0 | 93.8 | 6.2 | 0.0 |
| serum | | | | | | |
| albumin | | | | | | |
| Arginine | 12.9 | 498.6 | 5131.0 | 81.3 | 16.9 | 1.8 |
| Carbopol® | NA - precipitation of the protein | | | | | |
| 974P | | | | | | |
| Carbopol® | NA - precipitation of the protein | | | | | |
| 971P | | | | | | |
| Carboxy | 20.0 | 2.4 | 0.0 | 91.0 | 9.0 | 0.0 |
| methyl- | | | | | | |
| Dextran | | | | | | |
| (CMD) | | | | | | |
| Chitosan | 14.5 | 725.9 | 0.0 | 73.2 | 26.8 | 0.0 |
| Glycine | 14.2 | 0.0 | 0.0 | 100.0 | 0.0 | 0.0 |
| Hydroxy- | 14.3 | 2.1 | 5093.0 | 87.9 | 9.0 | 3.1 |
| propyl- | | | | | | |
| beta- | | | | | | |
| cyclo- | | | | | | |
| dextrin | | | | | | |
| (HPBCD) | | | | | | |
| Inulin | NA - precipitation of the protein | | | | | |
| Lecithin | NA - precipitation of the protein | | | | | |
| Maltitol | NA - precipitation of the protein | | | | | |
| Mannitol | 13.6 | 5277.0 | 0.0 | 98.0 | 2.0 | 0.0 |
| Na2 EDTA | 13.4 | 726.0 | 419.9 | 92.4 | 2.9 | 2.6 |
| Pluronic | 12.3 | 349.8 | 71.7 | 72.3 | 20.1 | 7.6 |
| F127 | | | | | | |
| Poloxamer | 11.4 | 171.1 | 0.0 | 97.2 | 2.8 | 0.0 |
| 188 | | | | | | |
| Polysor- | 11.7 | 0.0 | 0.0 | 100.0 | 0.0 | 0.0 |
| bate 20 | | | | | | |
| (Tween 20) | | | | | | |
| Sodium | 12.0 | 0.0 | 0.0 | 100.0 | 0.0 | 0.0 |
| lauryl | | | | | | |
| sulphate | | | | | | |
| (SLS) | | | | | | |
| Sorbitol | 14.2 | 4969.0 | 0.0 | 98.0 | 2.0 | 0.0 |
| Control | 13.4 | 4273.0 | 723.9 | 95.1 | 4.3 | 0.6 |
| (protein | | | | | | |
| only) | | | | | | |

The DLS results confirm that the formulations comprising Carbopol (971P et 974P), inulin, lecithin, maltitol, do not allow the stability of the anti-TNFα antibody to be maintained.

The other formulations show a satisfactory preservation of the protein, particularly in the presence of the excipients CMD, chitosan, glycine or HPBCD.

### Results of the Functional Test

The formulations considered as compatible (“Good” results only) under SEC and DLS, are then tested for functional activity.

The results of the functional test are given in Table 4 below:

TABLE 4
Results of the functional test

| | | | Results of the functional test - |
|---|---|---|---|
| Excipient | | | 14 days at 40° C. |
| Arginine | 1.25 | mg/mL | Activity preserved |
| Carboxymethyl-Dextran | 20 | mg/mL | Activity preserved |
| (CMD) | | | |
| Chitosan | 1.25 | mg/mL | Activity preserved |
| Glycine | 1.25 | mg/mL | Activity preserved |
| Hydroxypropyl-beta- | 20 | mg/mL | Activity preserved |
| cyclodextrin (HPBCD) | | | |
| Mannitol | 20 | mg/mL | Activity preserved |
| Na2 EDTA | 1.25 | mg/mL | Activity preserved |
| Pluronic F127 | 1.25 | mg/mL | Activity preserved |
| Poloxamer 188 | 1.25 | mg/mL | Activity slightly reduced |
| Polysorbate 20 | 1.25 | mg/mL | Activity slightly reduced |
| Sorbitol | 20 | mg/mL | Loss of activity |

### Conclusion:

At T0, following the addition of the anti-TNFα antibody, the formulations comprising the excipients Carbopol 971P, Carbopol 974P, lecithin, inulin and maltitol are determined as incompatible with the anti-TNFα antibody, as a result of the formation of white flakes (flocculation of the formulation), indicating that the protein of interest and/or the protein and the excipient thereof have precipitated.

Analysis of the combined results of the SEC, SLS and activity demonstrates the compatibility of the anti-TNFα antibody with the excipients arginine, CMD, chitosan, glycine, HPBCD, mannitol, Na EDTA, Pluronic® F127 and that they are therefore suitable for the antibody formulation.

### Example 2: Spray Drying

In order to obtain a formulation in a dry form, different formulations are subjected to a spray drying test. The formulations tested are listed in the table:

TABLE 5
Formulations tested for spray drying

| Protein | Excipients |
|---|---|
| Anti-TNFα antibody | Without excipients |
| (0 mg/mL or 5 mg/mL) | HPBCD (20 mg/mL) |
| | CMD (20 mg/mL) |
| | HPBCD (10 mg/mL) + CMD (10 mg/mL) |
| | HPBCD (19 mg/mL) + EDTA (1 mg/mL) |
| | Polysorbate 20 (1 mg/mL) + |
| | EDTA (1 mg/mL) + |
| | Trehalose (18 mg/mL) |
| | Poloxamer 188 (P188) (0.05 mg/mL) + |
| | EDTA (1 mg/mL) + |
| | Trehalose (18.95 mg/mL) |
| | CMD (18.95 mg/mL) + Polysorbate |
| | 20 (0.05 mg/mL) + Arginine (1 mg/mL) |
| | CMD (18.95 mg/mL) + Polysorbate |
| | 20 (0.05 mg/mL) + Glycine (1 mg/mL) |
| | Chitosan (20 mg/mL) |

The test makes it possible to assess the formation of dry powder and the possible need for the addition of protectants (surfactants and/or amino acids) during the drying stage.

Spray drying of the placebo solutions (without the addition of anti-TNFα antibody) was optimized on a laboratory-scale spray dryer of the type B-290 (Buchi, Flawil, Switzerland).

The test was then carried out with the excipients and with addition of anti-TNFα antibody.

The particles dried by spray-drying are collected in a reservoir attached to a cyclone. After the process, the powder is cooled at ambient temperature and transferred to a glass flask.

The parameters of the process are as follows:
Inlet temperature of the drying gas: 100-150° C.
Outlet temperature of the drying gas: 50-80° C.
Supply flow rate: 1-50 g/min
Suction: 100%
Gas: air or nitrogen

### Analysis of the Reconstituted Powders:

The powders obtained have a residual moisture content less than 15% by weight.

The powders are then reconstituted in acetate buffer (500 μL per 5 mg of powder) in order to monitor any negative impact of the spray drying stage.

All the powders satisfactorily preserve the quantity and quality of the anti-TNFα antibody protein.

As the objective is to administer the composition comprising the excipients and the protein of interest orally, with release preferably in the gastro-intestinal tract, assessment of the dissolution of the sprayed powders was not regarded as a relevant criterion.

### Example 3: Formulations Suitable for Oral Administration

The 10 formulations of Example 2, selected beforehand as ensuring the stability of the anti-TNFα antibody and dried, were then tested for oral administration.

In particular, a test was carried out in a model mimicking the ascending colon (SHIME® System from ProDigest) in order to study the effect of each formulation.

After passing through the SHIME® system, the samples are subjected to a functional activity test according to the following protocol:

2×10⁵ Jurkat cells transfected to express the membrane TFNα are incubated with 100 μl of anti-TNF antibodies at different concentrations (0 to 100 μl/ml, final concentration) at 4° C. for 30 minutes. After washing the antibody, goat anti-Fc IgG coupled to phycoerythrin (100 μl diluted to 1:100) is added at 4° C. over 30 minutes. The cells are washed and the mean fluorescence intensity is measured by flow cytometry. A response curve is established with the reference antibody (anti-TNFα antibody in PBS buffer) in order to define the minimum antibody concentration giving the plateau. All the samples are tested at 0.125 μg/ml. The results are expressed as a percentage with respect to a reference and normalized.

Results

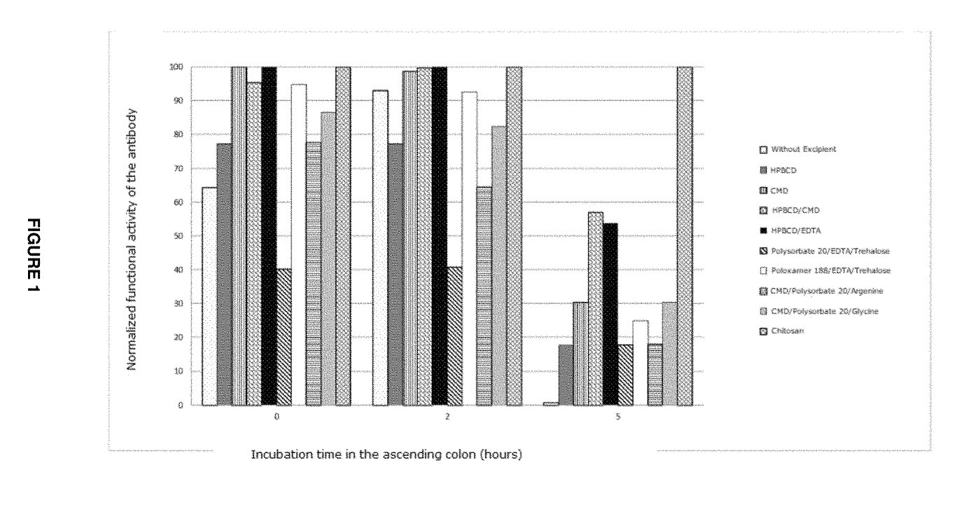

The results of the functional test at T0, T+2 hours and T+5 hours of incubation in the ascending colon are presented in FIG. 1.

All the samples preserve their ability to bind the membrane TNFα. The results confirm that the absence of excipients is deleterious for the preservation of the functional activity of the anti-TNFα antibody. In addition, the formulations comprising CMD, HPBCD+CMD, HPBCD+EDTA, CMD+polysorbate 20+glycine or chitosan show a satisfactory effect protecting the functional activity of the anti-TNFα antibody.

The test therefore confirms that formulations comprising the anti-TNFα antibody in the presence of the excipients CMD, chitosan, EDTA, glycine, HPBCD or polysorbate 20, alone or in combination, are therefore suitable for the antibody formulation for oral administration thereof.

### Example 4: Confirmation of the Formulations of Interest

The formulations of Examples 2 and 3 are then assessed with respect to their ability to preserve the integrity of the antibody and their non-toxicity for administration.

The following formulations are ruled out due to their potential side effects during oral administration, with targeted release in the gastro-intestinal tract:
the formulations comprising surface-active agents (polysorbate 20 or Poloxamer 188), as these, although perfectly tolerated for intravenous or subcutaneous administration, are suspected of being responsible, when ingested, for an increase in the risk of developing an inflammatory disease of the intestine such as Crohn's disease;
the formulations comprising molecules with anticoagulant effect, such as EDTA;
the formulations comprising polyols, in particular mannitol, maltitol, sorbitol, glycerol, lactitol or xylitol, which may have a laxative effect.

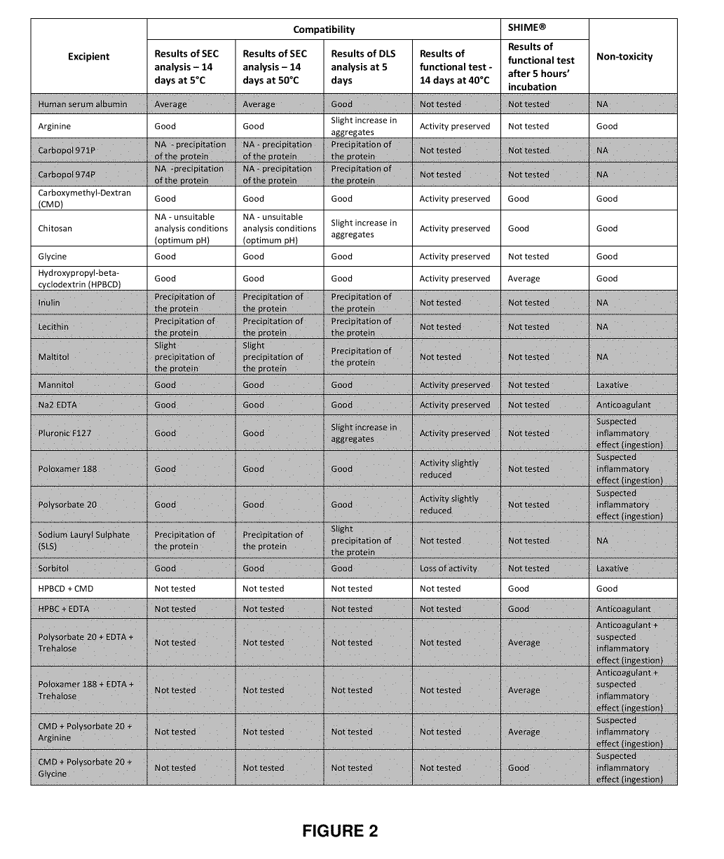

The characteristics of the different formulations tested are presented in the table in FIG. 2.

The greyed-out rows in the table in FIG. 2 represent the formulations not retained for oral administration of the anti-TNFα antibody.

The tests therefore confirm that formulations comprising the anti-TNFα antibody in the presence of the excipients CMD, chitosan, EDTA, or glycine, alone or in combination, are therefore suitable for the antibody formulation for oral administration thereof.

### Example 5: Oral Formulation with Release in the Gastro-Intestinal Tract

The following formulations:
anti-TNFα antibody+CMD
anti-TNFα antibody+CMD+HPBCD−
anti-TNFα antibody+CMD+HPBCD+glycine
anti-TNFα antibody+chitosan
   are then subjected to a coating step.

Different tests are carried out in order to test different coatings:
monolayer with one coating agent,
monolayer with a mixture of coating agents,
multilayer with one coating agent,
multilayer with several different coating agents,
multilayer with a mixture of coating agents,

The coating agents tested are preferentially enteric coatings due to the acid sensitivity of the antibody, in particular acrylic acid derivatives, methacrylic acid derivatives, ethyl acrylate derivatives, hydroxypropyl methylcellulose derivatives, polyvinyl acetate phthalate derivatives; in particular Eudragit® FS30D, Eudragit® S100, Eudragit® L100, Eudragit®L12.5, Eudragit® L30D-55, Eudragit® L100-55, Eudragit® S12.5, HPMC AS (AQOAT®), HPMCP®, Shellac.

The results confirm the satisfactory release of the anti-TNFα antibody.

1.-12. (canceled)

13. A pharmaceutical composition for oral administration, comprising a monoclonal anti-tumour necrosis factor alpha (TNFα) antibody, and a combination of carboxymethyl-dextran and cyclodextrin as pharmaceutically acceptable excipients.

14. A pharmaceutical composition according to claim 13, wherein the heavy chain of the anti-TNFα antibody comprises SEQ ID No.1 and wherein the light chain of the anti-TNFα antibody comprises SEQ ID No.2.

15. A pharmaceutical composition according to claim 13, wherein the anti-TNFα antibody is adalimumab.

16. A pharmaceutical composition according to claim 13, wherein the anti-TNFα antibody is produced in mammary epithelial cells from a non-human mammal or in the milk of a transgenic non-human mammal.

17. A pharmaceutical composition according to claim 13, further comprising an amino acid.

18. A pharmaceutical composition according to claim 13, wherein the composition is in solid form.

19. A pharmaceutical composition according to claim 18, wherein the composition in solid form is obtained by freeze drying, spray drying or by evaporation drying.

20. A pharmaceutical composition according to claim 13, wherein the composition is in the form of a sustained-release and/or delayed-release formulation.

21. A pharmaceutical composition with sustained release and/or delayed release according to claim 20, further comprising at least one coating agent.

22. A pharmaceutical composition with sustained release and/or delayed release according to claim 21, wherein the coating agent is selected from the group consisting of acrylic acid derivatives, methacrylic acid derivatives, ethyl acrylate derivatives, hydroxypropyl methyl cellulose derivatives, polyvinyl acetate phthalate derivatives; poly(methyl acrylate-co-methyl methacrylate-co-methacrylic acid, poly(methacrylate acid-co-methyl methacrylate), hydroxypropyl methylcellulose acetate succinate, and a mixture thereof.

23. A process for the preparation of a sustained-release and/or delayed-release pharmaceutical composition according to claim 13, said process comprising:
a) mixing the anti-TNFα antibody with a combination of carboxymethyl-dextran and cyclodextrin as pharmaceutically acceptable excipients,
b) drying, particularly by spraying, the mixture obtained in step a), and
c) coating the dried mixture with one or more coating agents.

24. The process of claim 23, wherein the drying is by spraying.

25. A method of treating an inflammatory disease or an auto-immune disease, comprising administering to a subject in need thereof the pharmaceutical composition of claim 13.


SEQUENCE LISTING (2 sequences)

SEQ ID NO: 1 - protein, 470 aa, Homo sapiens
MEFGLSWLFLVAILKGVQCEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 236 aa, Homo sapiens
MDMRVPAQLLGLLLLWLRGARCDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC